(19) United States
(12) Patent Application Publication
(10) Pub. No.: US 2026/0137415 A1
(43) Pub. Date: May 21, 2026

(54) SURGICAL INSTRUMENT

(71) Applicant: SPINAL SURGICAL STRATEGIES, INC., A NEVADA CORPORATION D/B/A KLEINER DEVICE LABS, Incline Village, NV (US)

(72) Inventors: Alan Burkholder, Denver, CO (US); Gregory Causey, Erie, CO (US); Lucas Simmons, Westminster, CO (US); Jeffrey Kleiner, Denver, CO (US); Edward John Grimberg, Golden, CO (US)

(73) Assignee: SPINAL SURGICAL STRATEGIES, INC., A NEVADA CORPORATION D/B/A KLEINER DEVICE LABS, Incline Village, NV (US)

(21) Appl. No.: 19/444,553

(22) Filed: Jan. 9, 2026

Related U.S. Application Data
(63) Continuation of application No. 18/693,795, now abandoned.
(60) Provisional application No. 63/247,256, filed on Sep. 22, 2021.

(51) Int. Cl. A61B 17/3207; A61B 17/00; A61B 17/16

(52) U.S. Cl. CPC A61B 17/320708; A61B 17/1671; A61B 2017/00473

(57) ABSTRACT

Surgical instruments that facilitate articulation of a scraping tool within a surgical space (e.g., a vertebrae), and are configured to remain locked in position when scraping and removing tissue. The surgical instruments include a handle and a retention shaft that insertably engages the handle. The retention shaft includes a split distal end having flexible first and second portions that include means for securing a removeable tip therebetween, the tip being configured to scrape/remove tissue and insertably engage the split distal end between the first and second portions. A pivot lock mechanism engages the handle and receives the retention shaft therein with means for moving same axially. The surgical instrument is moveable between an unlocked state in which the tip is moveable relative to the split distal end when secured therein, and a locked state in which the tip is not moveable relative to the split distal end when secured therein.

### CROSS-REFERENCE TO RELATED APPLICATIONS

This application is a continuation of U.S. patent application Ser. No. 18/693,795, filed on Mar. 20, 2024, which is a National Entry of PCT/US2022/044386, filed on Sep. 22, 2022, which claims the benefit of Provisional Application No. 63/247,256, filed on Sep. 22, 2021, which applications are hereby incorporated herein by reference.

### TECHNICAL FIELD

Embodiments of the invention relate generally to surgical instruments, and more particularly to articulatable surgical instruments for use in spinal and orthopedic procedures.

### BACKGROUND

Surgical instruments having the ability to articulate are advantageous in many types of procedures, including spinal and orthopedic procedures. Such surgical instruments include those designed to scrape and remove biological tissue from a patient, such as bone tissue in certain spinal and orthopedic procedures. Scraping tools that can be articulated within a surgical space (e.g., a patient's vertebrae in spinal surgery) after introduction through a narrow cannula allow a broader debridement geography. It is therefore desirable to provide improved articulatable surgical instruments that facilitate such placement of the scraping tool, and also remain locked in position when used in scraping and removing tissue from a patient, and thereby provide superior stability, patient outcomes and utility for surgeons.

### SUMMARY

The invention includes articulatable surgical instruments that facilitate placement and articulation of a scraping tool within a patient (e.g., in a surgical space such a vertebrae), and are also configured to remain locked in position when used in scraping and removing tissue from the patient.

One aspect of this invention relates to a surgical instrument that includes a handle having a proximal end and a distal end including a cannula; a retention shaft configured to insertably engage a distal end of the cannula, the retention shaft including a split distal end having flexible first and second portions; a removeable tip configured to insertably engage the split distal end between the first and second portions, wherein inner surfaces of the first and second portions include means for securing the removeable tip therebetween; and a pivot lock mechanism configured to operably engage the proximal end of the handle, and including a lock lever, a linkage member operably connected to the lock lever, and a sliding member operably connected to the linkage member. The linkage member is configured to translate rotational motion of the lock lever to linear motion of the sliding member, and the sliding member is configured to receive the retention shaft therein and move axially within the pivot lock mechanism. The surgical instrument is moveable between an unlocked pivotable articulation state in which the split distal end is a first distance from the cannula and the removeable tip is moveable relative to the split distal end when secured therein, and a locked articulation state in which the split distal end is a second distance from the cannula and the removeable tip is not moveable relative to the split distal end when secured therein, the second distance being less than the first distance.

Another aspect of this invention relates to a surgical instrument that includes a handle having a proximal end and a distal end including a cannula; a retention shaft configured to insertably engage a distal end of the cannula, the retention shaft including a split distal end having a first portion including a first inner surface having a first set of teeth and a second portion including a second inner surface having a second set of teeth; a removeable tip configured to insertably engage the split distal end between the first and second portions, and having a distal end and a proximal end including a third set of teeth on a first surface thereof configured to engage the first set of teeth and a fourth set of teeth on a second surface thereof configured to engage the second set of teeth; and a pivot lock mechanism configured to operably engage the proximal end of the handle, and including a lock lever, a linkage member operably connected to the lock lever, and a sliding member operably connected to the linkage member. The linkage member is configured to translate rotational motion of the lock lever to linear motion of the sliding member, and the sliding member is configured to receive the retention shaft therein and move axially within the pivot lock mechanism. The surgical instrument is moveable between an unlocked pivotable articulation state in which the split distal end is a first distance from the cannula and the removeable tip is moveable relative to the split distal end, and a locked articulation state in which the split distal end is a second distance from the cannula and the removeable tip is not moveable relative to the split distal end, the second distance being less than the first distance.

Still another aspect of this invention includes a surgical kit. The kit includes a surgical instrument having a handle with a proximal end and a distal end including a cannula; a retention shaft configured to insertably engage a distal end of the cannula, the retention shaft including a split distal end having flexible first and second portions; and a pivot lock mechanism configured to operably engage the proximal end of the handle, and including a lock lever, a linkage member operably connected to the lock lever, and a sliding member operably connected to the linkage member, wherein the linkage member is configured to translate rotational motion of the lock lever to linear motion of the sliding member, and the sliding member is configured to receive the retention shaft therein and move axially within the pivot lock mechanism. The kit also includes a plurality of interchangeable removeable tips configured to insertably engage the split distal end between the first and second portions, wherein inner surfaces of the first and second portions include means for securing the removeable tip therebetween. The surgical instrument is moveable between an unlocked pivotable articulation state in which the split distal end is a first distance from the cannula and one of the interchangeable removeable tips is moveable relative to the split distal end when inserted therein, and a locked articulation state in which the split distal end is a second distance from the cannula and one of the interchangeable the removeable tips is not moveable relative to the split distal end when inserted therein, the second distance being less than the first distance.

The details of one or more examples of the invention are set forth in the description below. Other features, objects, and advantages of the invention will be apparent from the detailed description of the examples and also from the drawing and the appending claims.

### BRIEF DESCRIPTION OF THE DRAWINGS

Embodiments of the invention are further described but are in no way limited by the following illustrations.

FIG. 1 is a top perspective view of a handle and cannula of a surgical instrument according to a first embodiment of the present invention;

FIG. 2 is a cross-sectional view of the handle and cannula of FIG. 1, taken along line 2-2;

FIG. 3 is a top perspective view of a pivot lock mechanism of the surgical instrument according to the first embodiment of the present invention;

FIG. 4 is a cross-sectional view of the pivot lock mechanism of FIG. 3, taken along line 4-4;

FIG. 5 is a top perspective view of a retention shaft of the surgical instrument according the first embodiment of the present invention;

FIG. 6 is a top plan view of a split distal end of the retention shaft of FIG. 5;

FIG. 7 is a side elevational view of the split distal end of the retention shaft of FIG. 5;

FIG. 8 is a top perspective view of the split distal end of the retention shaft of FIGS. 6 and 7 engaging a first embodiment of a removeable tip;

FIG. 9 is a top plan view of the split distal end of the retention shaft and removable tip of FIG. 8;

FIG. 10 is a top plan view of the split distal end of the retention shaft and removable tip of FIGS. 8 and 9, with the removeable tip shown pivoted in a first position;

FIG. 11 is a top perspective view of the split distal end of the retention shaft and removable tip of FIGS. 8-10, with the removeable tip shown pivoted in a first position;

FIG. 12 is a top plan view of the split distal end of the retention shaft and removable tip of FIGS. 8-11, with the removeable tip shown further pivoted in the first position;

FIG. 13 is a top plan view of the split distal end of the retention shaft and removable tip of FIGS. 8-12, with the removeable tip shown pivoted in a second position;

FIG. 14 is a top perspective view of the surgical instrument of the first embodiment of the present invention with the removeable tip in a pivotable state;

FIG. 15 is a side elevational view of the surgical instrument of FIG. 14, with the removeable tip shown pivoted in a first pivoted position;

FIG. 16 is a side elevational view of the surgical instrument of FIG. 14, with the removeable tip shown in a neutral (non-pivoted) position;

FIG. 17 is a top perspective view of the split distal end of the retention shaft of FIG. 7 engaging the removeable tip, with the removable tip shown in a locked neutral position;

FIG. 18 is a top plan view of the split distal end of the retention shaft and locked removable tip of FIG. 17;

FIG. 19 is a top perspective view of the split distal end of the retention shaft of FIG. 7 engaging the removeable tip, with the removable tip shown in a locked first pivoted position;

FIG. 20 is a top plan view of the split distal end of the retention shaft and removeable tip of FIG. 19;

FIG. 21 is a top perspective view of the surgical instrument of FIG. 14 with the removeable tip in a locked state;

FIG. 22 is a side elevational view of the surgical instrument of FIGS. 14 and 21, with the removeable tip shown locked in a neutral (non-pivoted) position;

FIG. 23 is a side elevational view of the surgical instrument of FIGS. 14 and 21, with the removeable tip shown locked in a first pivoted position;

FIG. 24 is a cross-sectional view of the pivot lock mechanism of FIG. 3, shown in an articulation pivotable position, with the proximal handle and retention shaft of the surgical instrument;

FIG. 25 is a top plan view of the split distal end of the retention shaft and removeable tip shown when the pivot lock mechanism is in the articulation pivotable position of FIG. 24;

FIG. 26 is a side elevational view of the split distal end of the retention shaft and removeable tip shown when the pivot lock mechanism is in the articulation pivotable position of FIG. 24;

FIG. 27 is a cross-sectional view of the pivot lock mechanism of FIG. 3, shown in an articulation locked position, with the proximal handle and retention shaft of the surgical instrument;

FIG. 28 is a top plan view of the split distal end of the retention shaft and removeable tip shown when the pivot lock mechanism is in the articulation locked position of FIG. 27;

FIG. 29 is a side elevational view of the split distal end of the retention shaft and removeable tip shown when the pivot lock mechanism is in the articulation locked position of FIG. 27;

FIG. 30 is a cross-sectional view of the pivot lock mechanism of FIG. 3, shown in a removeable tip release position, with the proximal handle and retention shaft of the surgical instrument;

FIG. 31 is a top plan view of the split distal end of the retention shaft and removeable tip shown when the pivot lock mechanism is in the removeable tip release position of FIG. 30;

FIG. 32 is a side elevational view of the split distal end of the retention shaft and removeable tip shown when the pivot lock mechanism is in the removeable tip release position of FIG. 30;

FIG. 33 is a cross-sectional view of the pivot lock mechanism of FIG. 3, shown in a retention shaft release position, with the proximal handle and retention shaft of the surgical instrument;

FIG. 34 is a top plan view of the split distal end of the retention shaft and removeable tip shown when the pivot lock mechanism is in the retention shaft release position of FIG. 33;

FIG. 35 is a side elevational view of the split distal end of the retention shaft and removeable tip shown when the pivot lock mechanism is in the retention shaft release position of FIG. 33;

FIG. 36 is a top perspective view of an embodiment of a removeable tip of the surgical instrument of the first embodiment of the present invention that has a stirrup configuration;

FIG. 37 is a top perspective view of another embodiment of a removeable tip of the surgical instrument of the first embodiment of the present invention that has a cup curette configuration;

FIG. 38 is a side elevational view of the removeable tip of FIG. 37;

FIG. 39 is a top plan view of the removeable tip of FIG. 37;

FIG. 40 is a partial top perspective view of a first embodiment of a release mechanism for the retention shaft of the surgical instrument of FIGS. 14 and 21;

FIG. 41 is a side elevational view of the surgical instrument of the first embodiment of the present invention and the retention shaft release mechanism of FIG. 40;

FIG. 42 is a partial top perspective view of the retention shaft release mechanism of FIG. 40, as deployed by a user;

FIG. 43 is a side elevational view of the surgical instrument of the first embodiment of the present invention and the deployed retention shaft release mechanism of FIG. 42;

FIG. 44 is a partial environmental view of the surgical instrument according to a second embodiment of the present invention in use, with the removeable tip shown in a first pivoted position;

FIG. 45 is a partial environmental view of the surgical instrument of the surgical instrument of FIG. 44 in use, with the removeable tip shown in a second pivoted position;

FIG. 46 is a detailed partial top perspective view of the surgical instrument of FIG. 44, with the removeable tip shown in a pivoted position;

FIG. 47 is a top partial plan view of the surgical instrument of FIG. 45;

FIG. 48 is a side elevational view of a surgical instrument according to a third embodiment of the present invention, with a release mechanism for a retention shaft shown in a locked position;

FIG. 49 is a front view of the surgical instrument of FIG. 48;

FIG. 50 is a side elevational view of the engaged retention shaft and removeable tip of the surgical instrument of FIG. 48;

FIG. 51 is a top plan view of the retention shaft and removeable tip shown in FIG. 50;

FIG. 52 is a side elevational view of the surgical instrument of FIG. 48, with the release mechanism for the retention shaft shown in an unlocked position;

FIG. 53 is a front view of the surgical instrument of FIG. 52;

FIG. 54 is a side elevational view of the disengaged retention shaft and removeable tip of the surgical instrument according to the third embodiment of the present invention;

FIG. 55 is a top plan view of the retention shaft and removeable tip shown in FIG. 54;

FIG. 56 is a side elevational view of the surgical instrument of FIG. 48, with the release mechanism for the retention shaft shown in a deployed/slide position that allows release of the retention shaft;

FIG. 57 is a front view of the surgical instrument shown in FIG. 56;

FIG. 58 is a side elevational view of the split distal end of the retention shaft;

FIG. 59 is a top plan view of the split distal end of the retention shaft shown in FIG. 58;

FIG. 60 is an exploded top perspective view of another embodiment of a retention shaft and removeable cup curette tip of the surgical instrument according to the third embodiment of the present invention;

FIG. 61 is an exploded side elevational view of the split distal end of the retention shaft and removeable tip of FIG. 60;

FIG. 62 is an exploded top plan of the split distal end of the retention shaft and removeable tip of FIG. 60;

FIG. 63 is an exploded partial top perspective view of the retention shaft and removeable tip of FIG. 60;

FIG. 64 is an exploded partial top perspective view of the retention shaft and removeable tip of FIGS. 60 and 63, with a portion of the removeable tip removed for clarity and illustration;

FIG. 65 is a top perspective view of the removeable tip shown in FIGS. 60-62 ;

FIG. 66 is a top plan view of the removeable tip of FIG. 65;

FIG. 67 is a side elevational view of the removeable tip of FIG. 65;

FIG. 68 is a top perspective view of a removeable stirrup tip;

FIG. 69 is a top plan view of the removeable tip of FIG. 68;

FIG. 70 is a side elevational view of the removeable tip of FIG. 68;

FIG. 71 is a detailed side elevational view of a removeable tip according to another embodiment of the invention; and

FIG. 72 is a top plan view of the removeable tip of FIG. 71.

### DETAILED DESCRIPTION OF ILLUSTRATIVE EMBODIMENTS

This invention includes embodiments of an articulatable surgical instrument having a handle, a cannula, a pivot lock mechanism, a retention shaft, and an interchangeable/removable tip configured to releasably and operationally engage the retention shaft and scrape/remove tissue from a patient during surgery, as further discussed below.

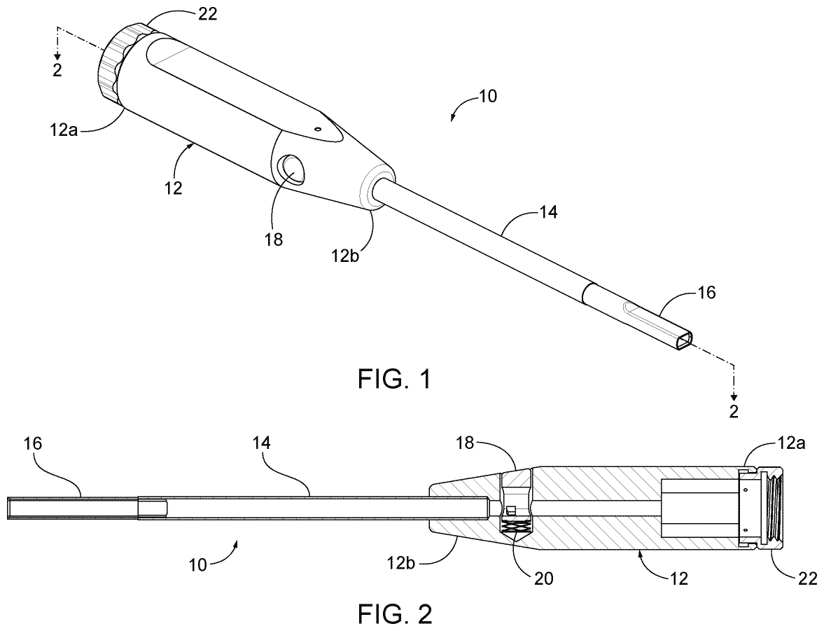

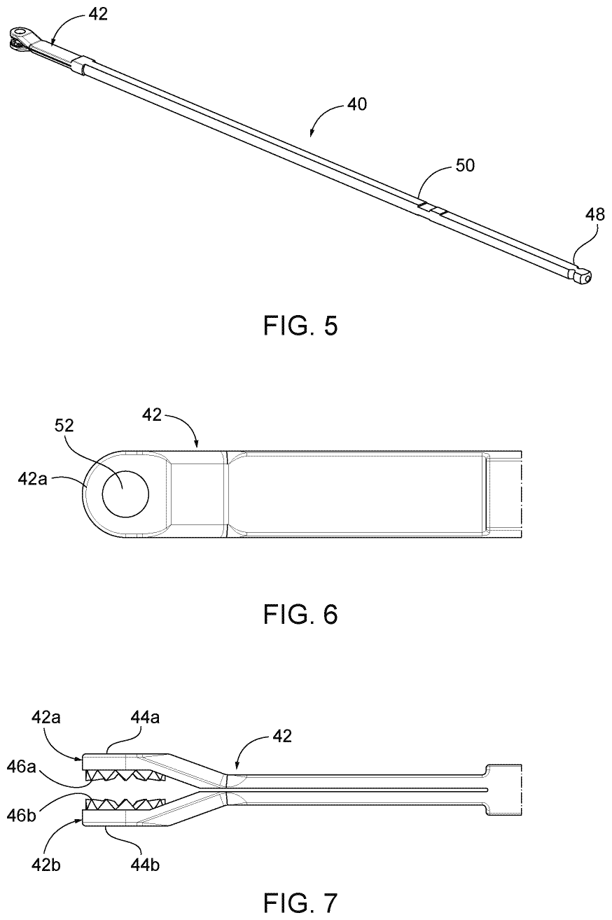

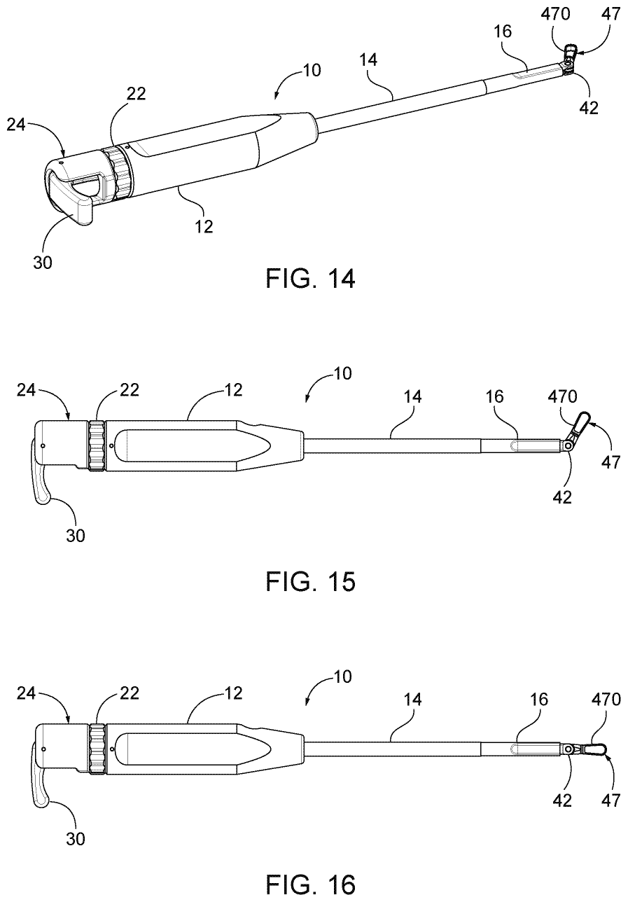

A first embodiment of the surgical instrument 10 is shown in FIGS. 14-16 and 21-23. Referring to FIGS. 1 and 2, the surgical instrument 10 includes a handle 12 having opposed proximal and distal ends 12a, 12b. A cannula 14 is operably connected to the distal end 12b. The open cannula 14 allows for passage of a retention shaft 40 (see FIG. 5) through the cannula 14 along its length, as further discussed below.

The cannula 14 includes a locking interface 16 on its distal end that is configured to interact with the retention shaft 40 to hold a portion thereof (e.g., via interlocked teeth or other means) in position with one of the removeable tips, as further discussed below. Articulation of the surgical instrument 10 may be locked or unlocked when the locking interface 16 is in contact with the retention shaft 40.

The handle 12 includes a retention shaft release button 18 and an associated retention shaft release spring 20. When pressed, the release button 18 allows the retention shaft 40 to be released from the cannula 14 of the handle 12 and pivot lock mechanism, as further discussed below, thereby preventing the retention shaft 40 from being released unintentionally. The handle 12 also includes a handle collar 22 at its proximal end 12a that is configured to attach the handle 12 to the pivot lock mechanism.

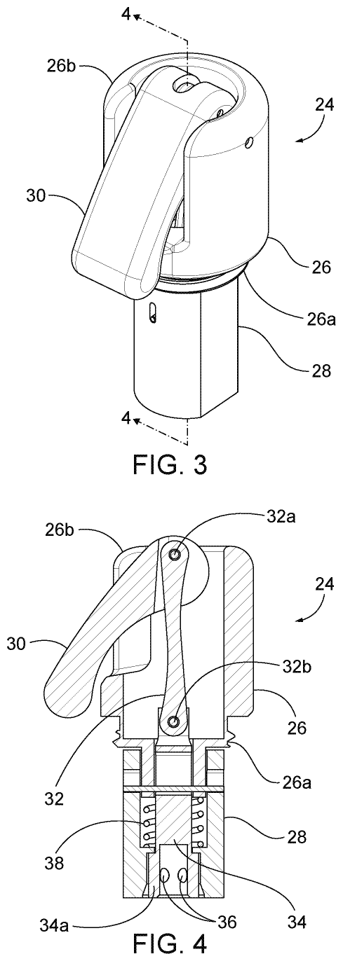

The pivot lock mechanism 24 is shown in FIGS. 3 and 4 and will now be described. The pivot lock mechanism 24 is configured to operably engage the handle 12 (e.g., via the handle collar 22, or directly in other embodiments), and includes a lock housing 26, a release collar 28 extending from a first end 26a of the lock housing 26, and a lock lever 30 extending from and rotatably engaging a second end 26b of the lock housing 26. The lock lever 30 provides a means for user actuation to lock and unlock passive articulation of removeable tips engaged with a distal end of the retention shaft, as further discussed below. The pivot lock mechanism 24 further includes a linkage member 32 contained in the lock housing 26 and a sliding member 34 contained in the release collar 28 and operably connected to the linkage member 32. More particularly, a first end of the linkage member 32 is connected to the lock lever 30 by a first connector 32a, and a second end of the linkage member 32 is connected to the sliding member 34 by a second connector 32b. The linkage member 32 is configured to translate rotational motion of the lock lever 30 to linear motion of the sliding member 34. The sliding member 34 includes a distal portion 34a that is configured to receive the retention shaft 40 therein. As further described below, sliding member 34 moves linearly/axially with respect to the release collar 28 in the lock housing 26.

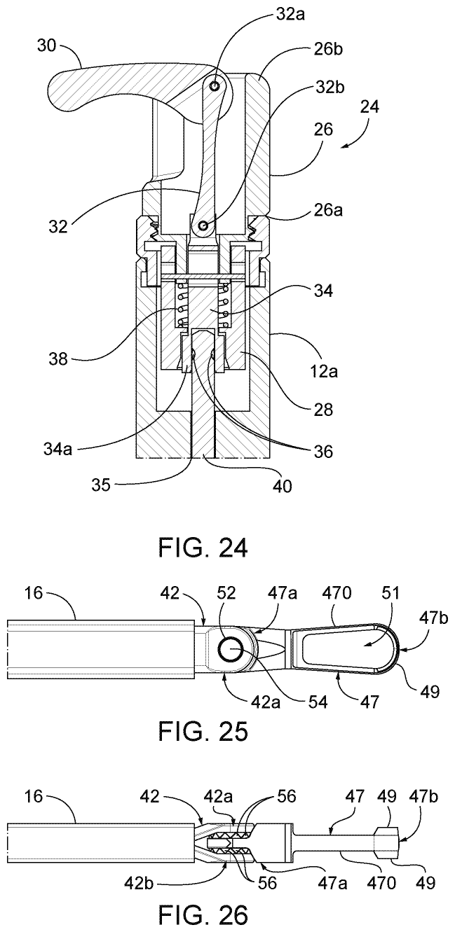

The handle 12 further includes an opening 35 within its proximal end 12a, as shown in FIGS. 24, 27, 30 and 33. The opening 35 is configured to receive the retention shaft 40 therethrough and ensures that the retention shaft 40 is centered within the sliding member 34 and the handle 12.

The release collar 28 contains a pivot lock spring 38 disposed about the sliding member 34. The spring 38 is configured to keep the release collar 28 and sliding member 34 in the same relative position until the lock lever 30 is moved into the release position. Slots (not shown) may be formed in the release collar 28 to restrict its further movement as the sliding member 34 continues to advance, releasing the retention shaft 40.

The sliding member 34 also houses a plurality of retention balls 36 that are configured to retain the retention shaft 40. As shown in FIGS. 24, 27, 30 and 33, the retention balls 36 are forced inward by the release collar 28 and in that position engage with a groove in the retention shaft 40. When the release collar 28 is retracted relative to the sliding member 34, the retention balls 36 move radially outward, which in turn releases the retention shaft 40. The retention balls 36 are still captured by the release collar 28 as the translation of the release collar 28 is limited. The pivot lock spring 38 also helps to maintain the retention balls 36 in position, and therefore keeps the retention shaft 40 locked in.

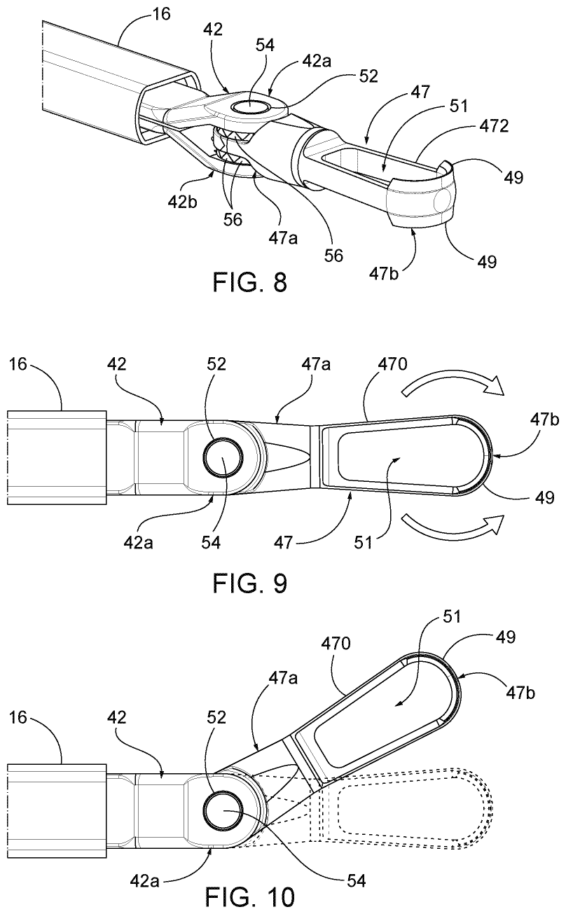

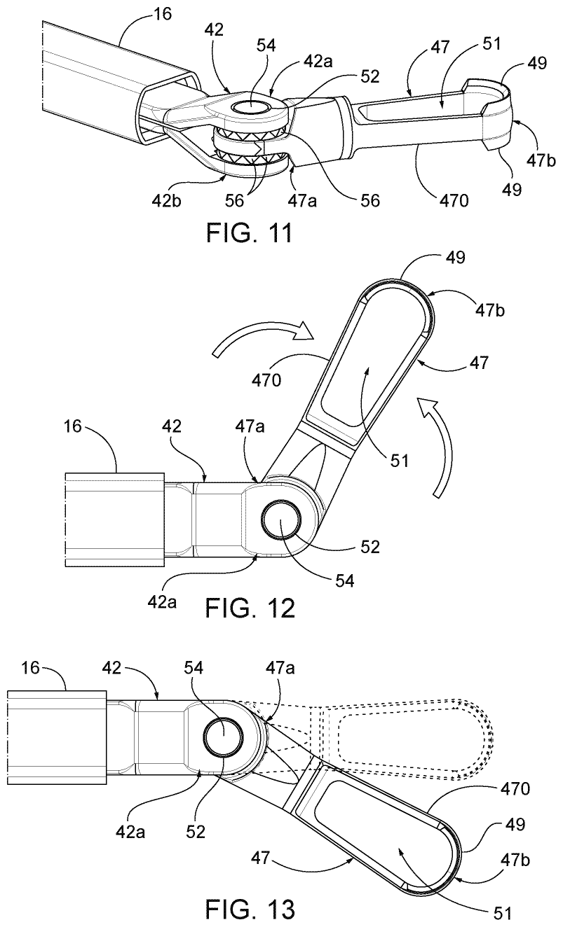

Embodiments of the retention shaft 40 and components thereof are shown in FIGS. 5-13 and will now be described. Referring to FIGS. 5 and 7, the retention shaft 40 includes a split (i.e., Y-shaped) distal end 42 having first and second portions 42a, 42b that are mirror images of each other. Each of the first and second portions 42a, 42b includes an outer surface 44a, 44b, respectively, and an inner surface 46a, 46b, respectively. The inner surfaces 46a, 46b each have teeth formed thereon. The split distal end 42 is configured to be flexible so that the first and second portions 42a, 42b can be separated and splay open to accept an interchangeable removeable tip 47 between them, and the teeth on the inner surfaces 46a, 46b are configured to index and lock the articulation of the tip 47 about a pin axis, as shown in FIGS. 8-13 and further described below. In other embodiments, the removeable tip 47 can be secured between the first and second portions 42a, 42b by a friction fit.

With continued reference to FIG. 5, the retention shaft 40 also includes a proximal end 48. As further discussed below, the proximal end 48 is configured to be inserted into the sliding member 34 of the pivot lock mechanism 24 and retained therein.

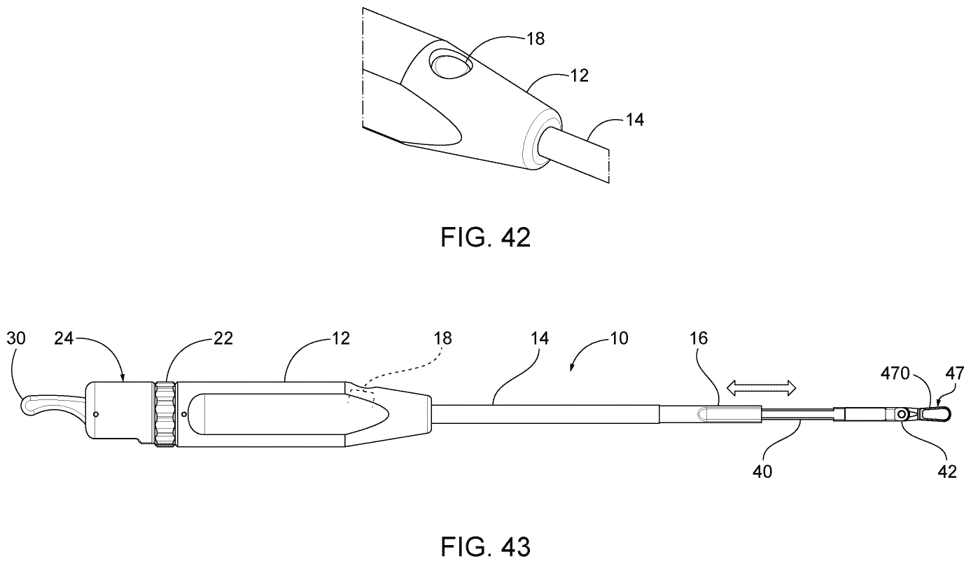

The retention shaft 40 also includes an intermediate portion 50 that extends between the split distal end 42 and proximal end 48 and has additional features that facilitate securing the retention shaft 40 within the cannula 14 of the surgical instrument 10. These features of the retention shaft 40 are designed to engage with the handle push button 18 (see FIGS. 1, 2 and 40-43) and only allow limited motion of the retention shaft 40 relative to the handle 12, and therefore the lock lever 30 until the button 18 is pushed. Once the button 18 is pushed, the retention shaft 40 is free to translate when the lock lever 30 is moved to the release position. The proximal end 48 and intermediate portion 50 of the retention shaft 40 are passed through the cannula 14, into the handle 12 into the pivot lock mechanism 24 (via its proximal end 12a). The proximal end 48 of the retention shaft 40 is configured to transmit linear/axial motion of (i.e., linear/axial motion of the sliding member 34) that engages the proximal end 12a of the handle 12. More particularly, movement of the retention shaft 40 in a proximal direction, as generated by the linear/axial motion of the pivot lock mechanism 24, causes the split distal end 42 to move in the proximal direction and contact the locking interface 16 of the cannula 14 and the teeth on the interior surfaces 46a, 46b of the split distal end 42 to engage the removeable tip 47 positioned therebetween to prevent rotation thereof and place the surgical instrument in a locked articulation state, as further discussed below. Movement of the retention shaft 40 in a distal direction causes the split distal end 42 to splay open, separating first and second portions 42a, 42b such that the tip 47 can be positioned at various angular orientations with respect to the split distal end 42 (see FIGS. 8-13), thereby placing the surgical instrument 10 in a pivotable articulation state, as further discussed below.

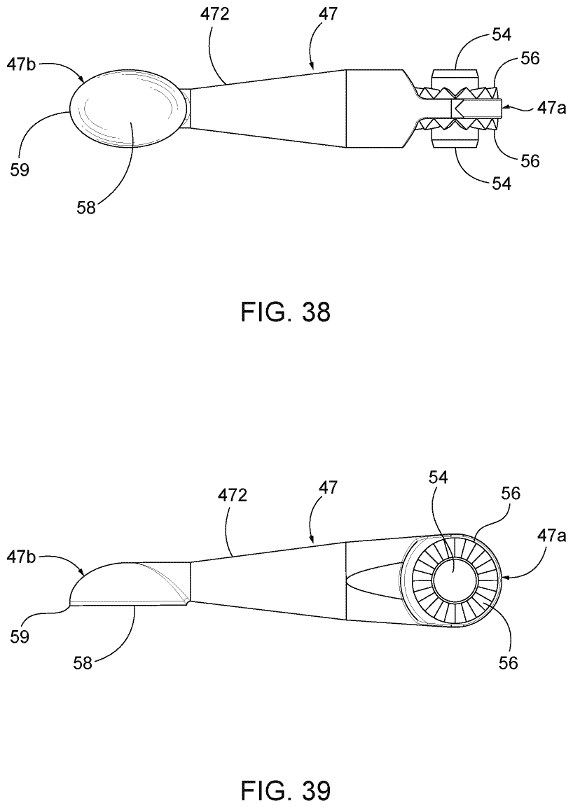

In order to facilitate engagement of the removeable tip 47 within the split distal end 42, the first and/or second portions 42a, 42b of the split distal end 42 include(s) an opening 52 that defines a pivot axis. The tip 47 includes proximal and distal ends 47a, 47b, with at least one axial member, or pin, 54 disposed at the proximal end 47a that is configured to insertably engage the opening(s) 52 of the split distal end 42, to further define the pivot axis about which the tip 47 is rotated/pivoted. As shown in FIGS. 8, 11, 17, 19, 26, 29, 32 and 36-39, the proximal end 47a of the tip 47 also includes teeth 56 arranged around the pin 54. The teeth 56 are configured to interdigitatably engage and interlock with the teeth on the interior surfaces 46a, 46b of the split distal end 42.

FIGS. 8-23 show the split distal end 42 of the retention shaft 40 engaging a first embodiment of the removeable tip 47 having a stirrup (i.e., open) configuration 470, including one or more cutting edges 49 (i.e., for scraping bone or other tissue during surgery) and an opening 51. FIGS. 8-13 show the split distal end 42 and the tip 47 in the pivotable (i.e., unlocked) articulation state of the surgical instrument 10, while FIGS. 14-16 show the entire surgical instrument 10 in this state. In the pivotable state, the retention shaft 40 is extended distally from the locking interface 16 of the cannula 14, which causes the split distal end 42 of the retention shaft 40 to splay open, separating first and second portions 42a, 42b enough that the tip 47 can be moved, rotated and positioned at various angular orientations with respect to the split distal end 42, as shown in FIGS. 9-13. FIGS. 14-16 show the entire surgical instrument 10 in the pivotable state, with the lock lever 30 of the pivot lock mechanism 24 in a first position that corresponds to the pivotable state. This first position of the lock lever 30 is also shown in FIG. 24, with FIGS. 25 and 26 showing the split distal end 42 and the tip 47 in the pivotable state as well.

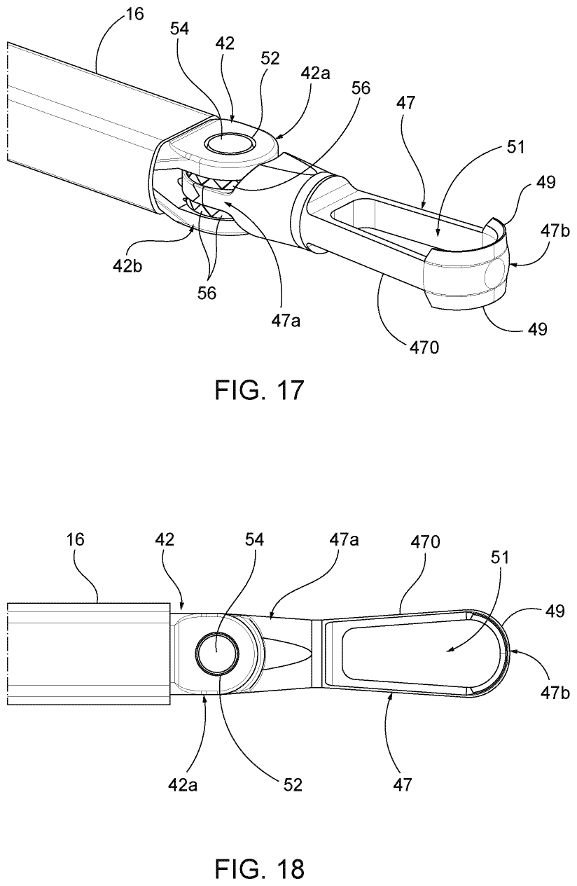

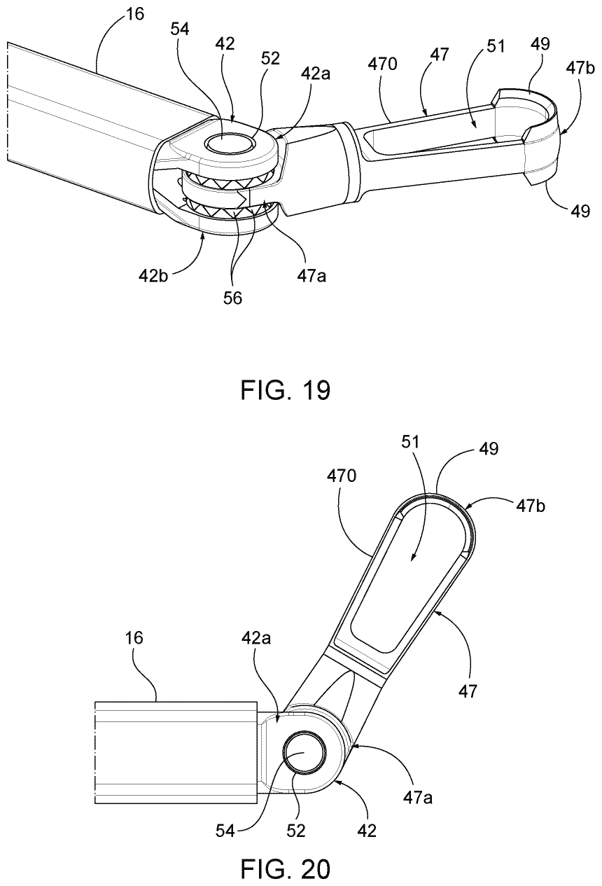

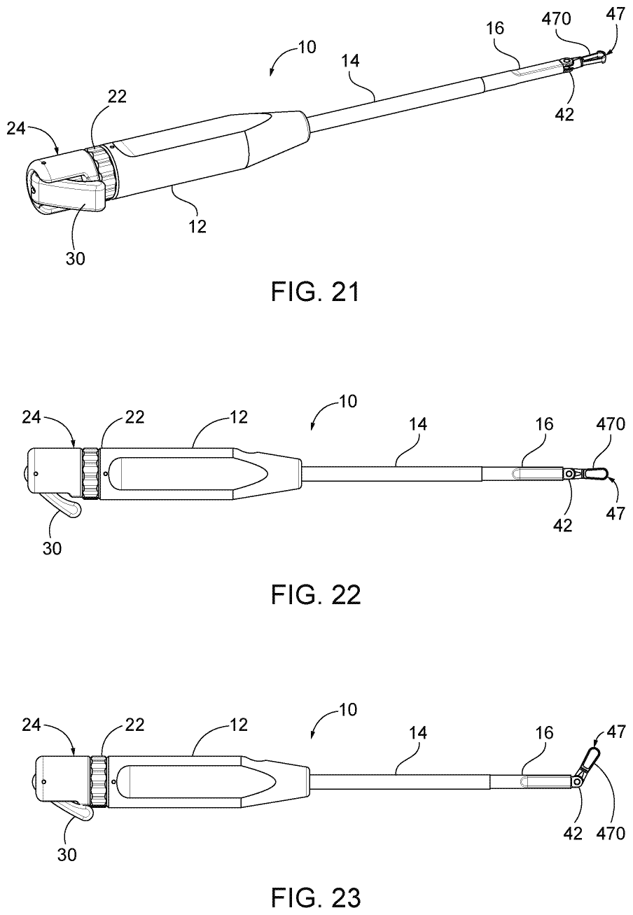

FIGS. 17-20 show the split distal end 42 and the tip 47 in the locked articulation state of the surgical instrument 10 (i.e., the tip 47 being unpivoted in FIGS. 17 and 18 and being pivoted at an angular orientation with respect to the split distal end 42 in FIGS. 19 and 20), while FIGS. 21-23 show the entire surgical instrument 10 in this state. In the locked state, the retention shaft 40 is moved in a proximal direction (i.e., farther into the cannula 14 and pivot lock mechanism 24), which causes the split distal end 42 to contact the locking interface 16 of the cannula 14, the first and second portions of distal end 42a, 42b to move closer to each other, and the teeth on the interior surfaces 46a, 46b of the first and second portions 42a, 42b to securely engage the teeth 56 of the removeable tip 47 to prevent its rotation or other movement. The tip 47 is thereby locked in place within the split distal end 42, rendering the surgical instrument 10 ready for use in a surgical procedure.

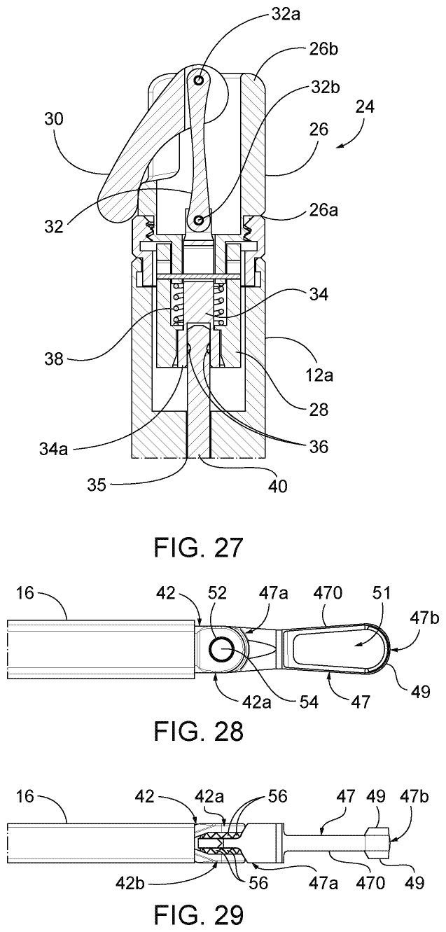

FIGS. 21-23 show the entire surgical instrument 10 in the locked state, with the lock lever 30 of the pivot lock mechanism 24 in a second position that corresponds to the locked state. This second position of the lock lever 30 is also shown in FIG. 27, with FIGS. 28 and 29 showing the split distal end 42 and the tip 47 in the locked state as well.

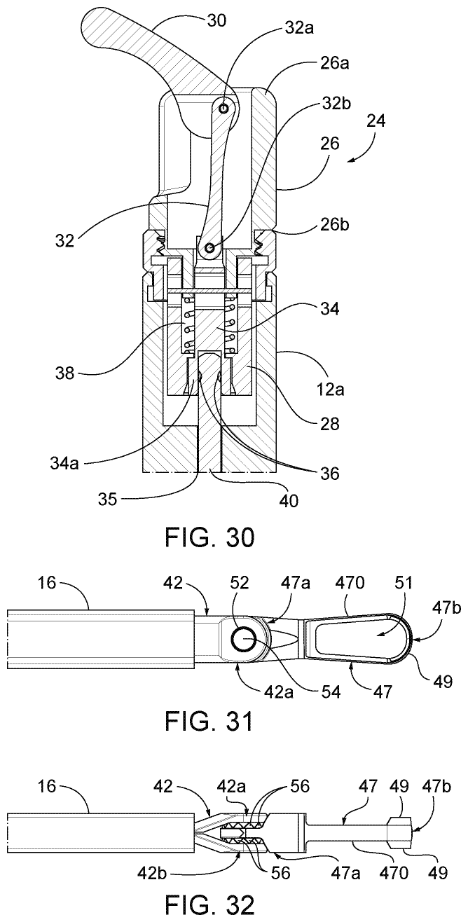

FIG. 30 shows the pivot lock mechanism 24 with its lock lever 30 in a third position that enables the release of the removeable tip 47 from the split distal end 42. In this position, the retention shaft 40 remains locked in place, but it has advanced far enough to allow the removeable tip 47 to articulate. In this position, the bottom surface of the release collar 28 is flush with the bottom surface of the sliding member 34. The retention shaft 40 is thereby kept locked in, but has advanced enough to allow the removeable tip 47 to articulate. FIGS. 31 and 32 show the split distal end 42 and the tip 47 in this release position.

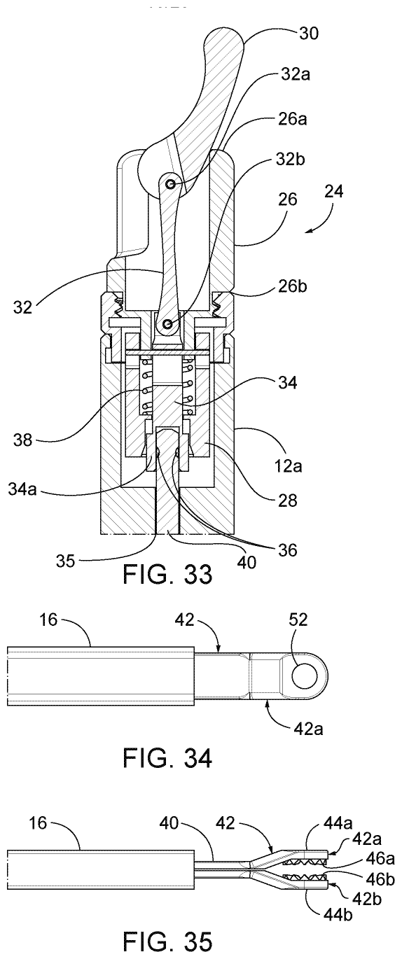

FIG. 33 shows the pivot lock mechanism 24 with its lock lever 30 in a fourth position that enables the release of the retention shaft 40 from the locking interface 16 of the cannula 14 (i.e., with distal axial movement of the sliding member 34). FIGS. 34 and 35 show the retention shaft 40 (including the split distal end 42 thereof) and the locking interface 16 of the cannula 14 in this release position.

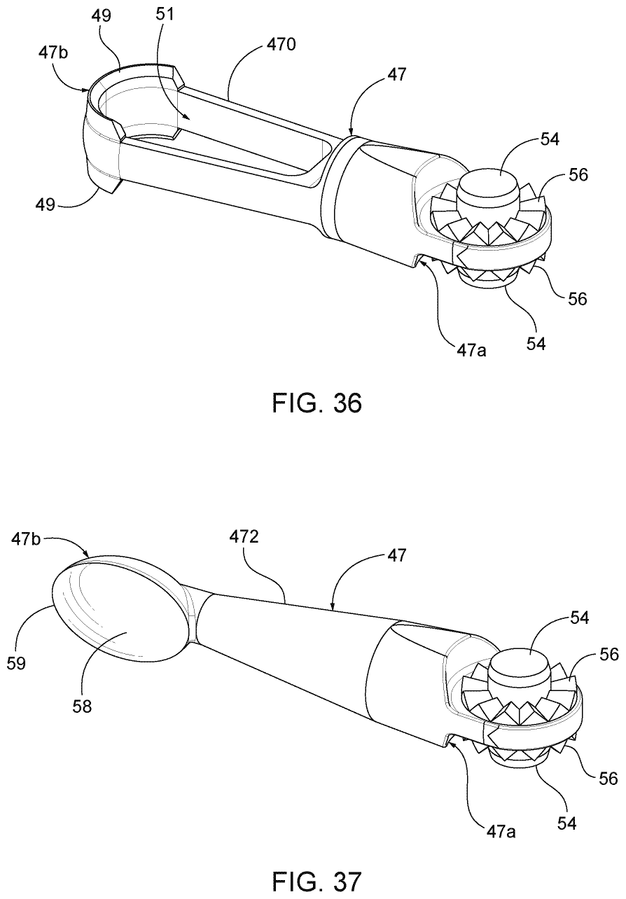

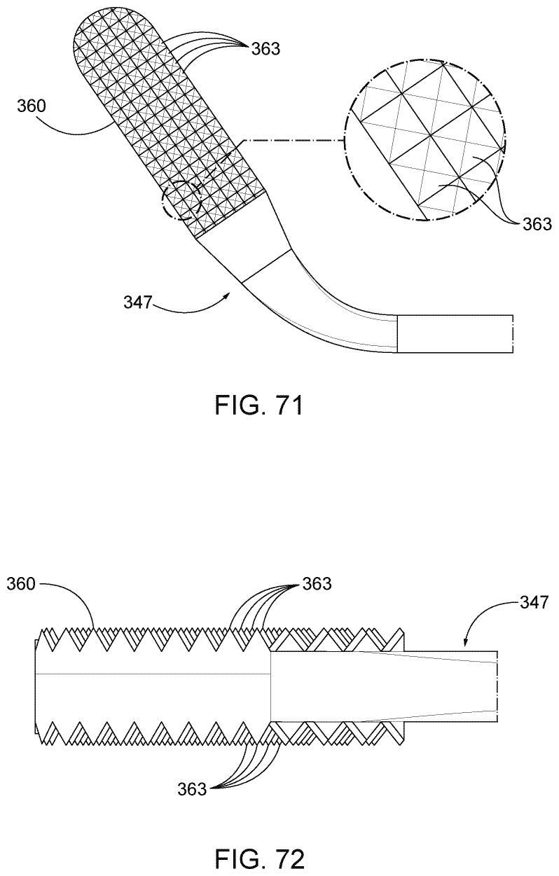

FIG. 36 shows removeable tip 47 having a stirrup (i.e., open) configuration 470, as shown and discussed above. Another embodiment of the removeable tip 47 is shown in FIGS. 37-39, wherein the tip 47 has a cup curette (i.e., closed, or solid) configuration 472 and includes a debridement feature 58 and a cutting edge 59 (i.e., for scraping bone or other tissue during surgery) at its distal end (opposite the pin 54 and teeth 56 thereof). Other configurations of the tip 47 are also possible, depending on the surgical procedure (i.e., see FIGS. 71-72, as further discussed below).

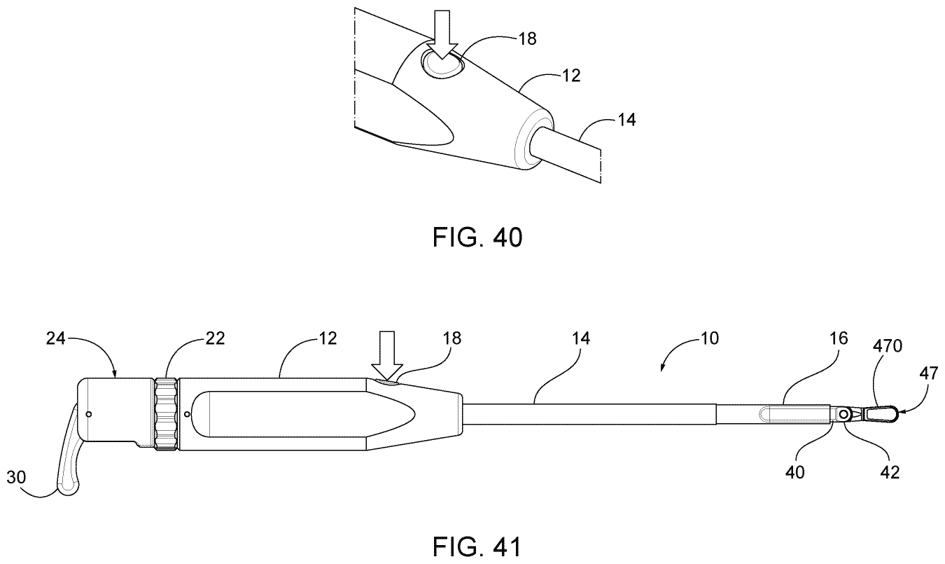

FIGS. 40-43 show the release mechanism for the retention shaft 40 in the first embodiment of the surgical instrument 10. The release mechanism includes a retention shaft release button 18 disposed within the handle 12, as also shown in FIGS. 1 and 2. Depressing the release button 18 (as shown by the arrows in FIGS. 40 and 41) releases the retention shaft 40, allowing it to move with respect to the cannula 14 and locking interface 16 thereof (as shown by the arrow in FIG. 43), which allows the retention shaft 40 to be fully removed from the handle 12.

The surgical instrument 10 also includes safety features for preventing accidental injury to a patient during use, such as the accidental release of the removeable tip 47 within a patient's vertebral disc space.

The release mechanism and method for removing tip 47 involves a series of steps that include releasing the lock lever 30 and pushing the tip 47 to fold up against the retention shaft 40. The lock lever 30 must be in the retention shaft release position for the retention shaft 40 to be removed, and in order to place the lock lever 30 in the release position, the button 18 must be depressed. Once the retention shaft 40 is removed from the handle 12, the tip 47 can be removed. This series of steps are required so that the retention shaft 40 is not inadvertently released from the handle 12 and the tip 47 is not released while a surgeon is working in the disc space.

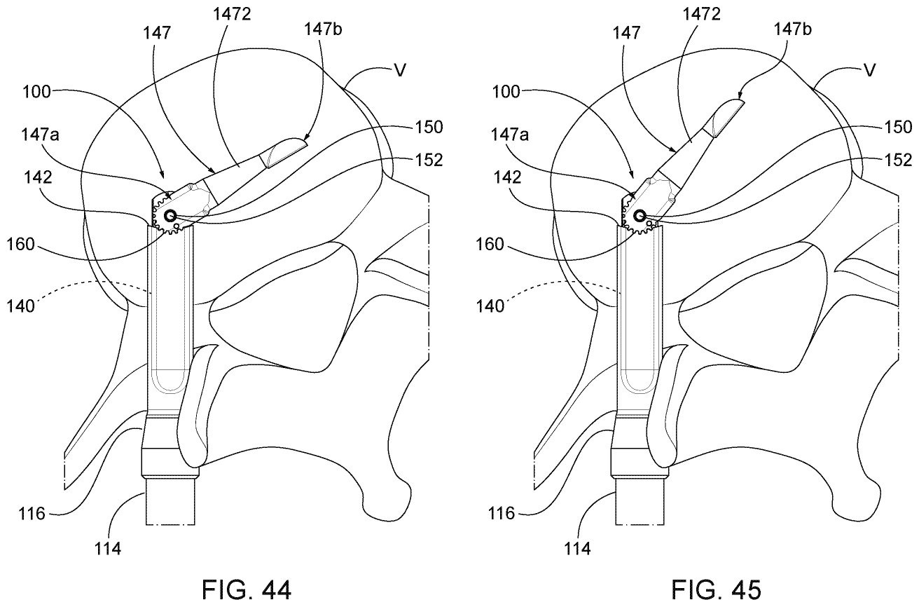

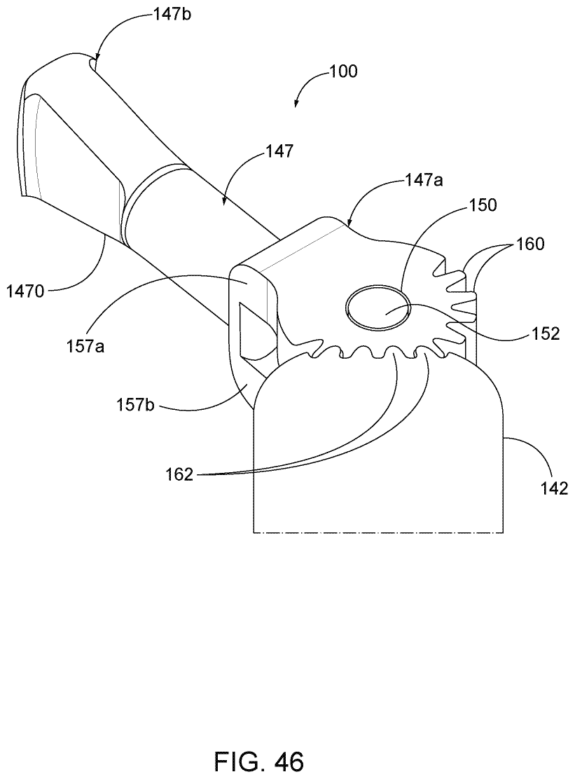

FIGS. 44-47 shows a second embodiment of the surgical instrument 100 of the present invention. Unless otherwise indicated, the structural and functional features and operation of this surgical instrument are the same as or similar to those described in connection with the first embodiment above. FIGS. 45 and 46 showing the surgical instrument 100 being used in a spinal surgical procedure (i.e., in a patient's vertebrae V), with the removeable tip 147 being shown in first and second pivoted positions within the vertebrae V. The removeable tip 147 includes proximal and distal ends 147a, 147b, and is specifically shown as a cup curette 1472 in FIGS. 44-45. The proximal end 147a of the tip 147 is split and includes first and second portions 157a, 157b, each of which has teeth 160 thereon and an opening 150 that defines a pivot axis. The proximal end 140 includes a distal end 142 that is configured to be inserted between the first and second portions 157a, 157b and includes a pin 152 that is configured to insertably and rotatably engage the openings 150 therein. The distal end 142 of the retention shaft 140 both having teeth 160, 162, on their respective outer surfaces and interdigitatably engaged/interconnected to one another to facilitate the pivoting motion of the tip 147 and the locking thereof in place (see FIG. 46). In other embodiments, the distal end 142 can be secured within the removeable tip 147 can be secured between the first and second portions 157a, 157b by a friction fit.

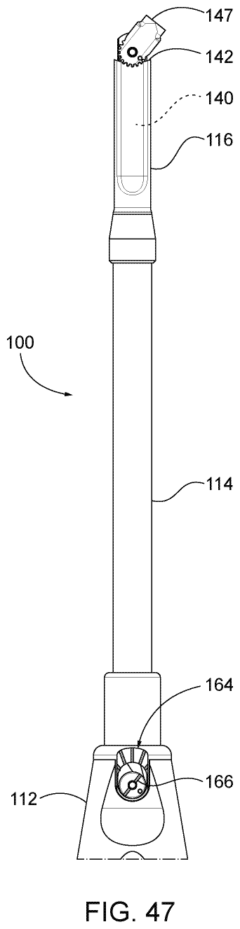

FIG. 47 shows the surgical instrument 100 with its handle 112, cannula 114, locking interface 116, retention shaft 140 and removeable tip 147, which is arranged in a first pivoted position. The handle 112 includes an angle indicator 164 with a dial 166 that is configured to indicate the angle of the tip 147. More particularly, as the tip 147 articulates, the dial 166 will rotate to give an indication of the orientation of the tip 147. The angle indicator 164 and dial 166 are located on the handle 112 so as to be outside of the surgical opening/wound to be visible to the surgeon/user when the tip 147 is in the opening/wound and may not be visible.

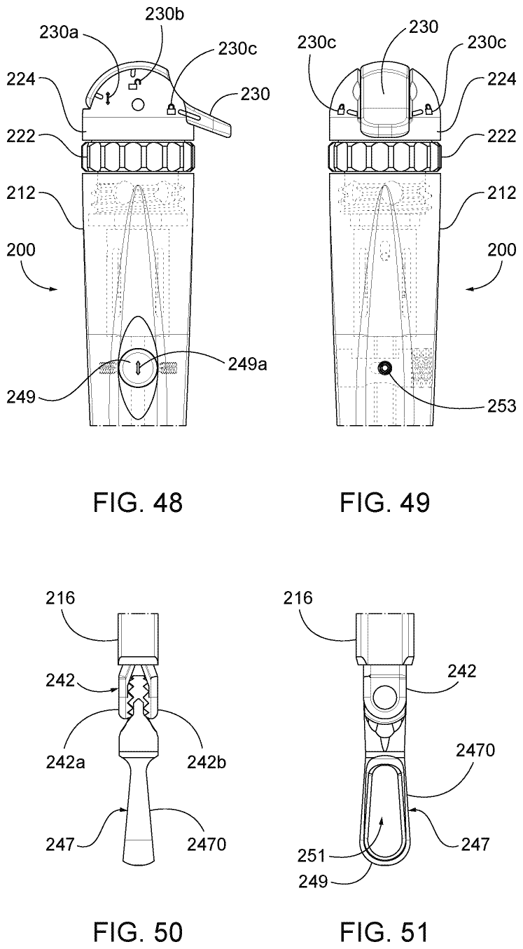

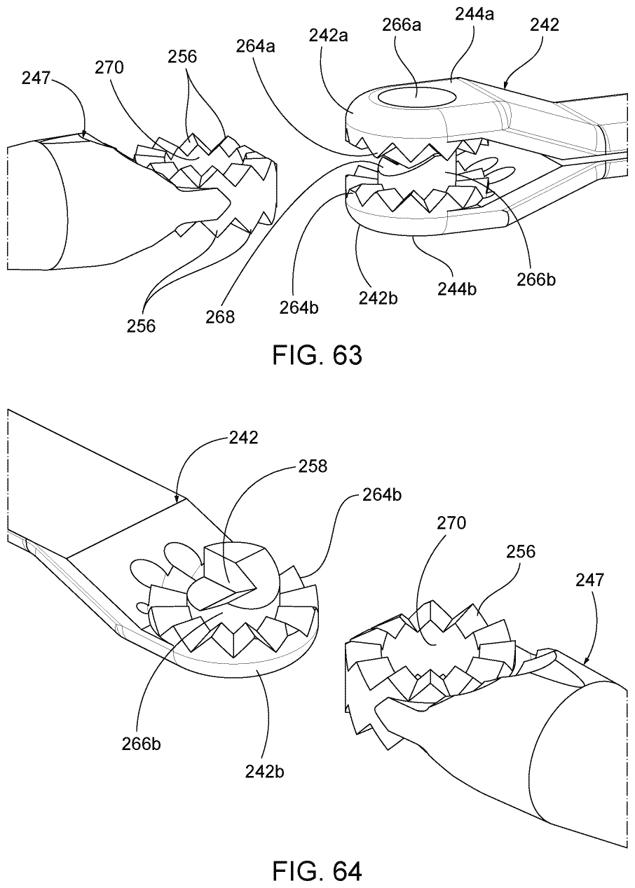

FIGS. 48-70 show the features of a third embodiment of the surgical instrument 200. Unless otherwise indicated, the structural and functional features and operation of this surgical instrument are the same as or similar to those described in connection with the first embodiment above.

In the third embodiment of the surgical instrument 200, the retention shaft does not include the same push button release feature as the previous embodiments. The release of the retentions shaft is accomplished with a similar push button (as the first embodiment), however the push button of the third embodiment either stops or allows the sliding member to advance. If the sliding member is not advanced, the retention shaft cannot be removed from the cannula. The retention shaft release mechanism of the third embodiment is further discussed below, with reference to FIGS. 48-59.

FIGS. 48 and 49 show a pivot lock mechanism 224 of the surgical instrument 200 with a lock lever 230 in a locked position, as denoted by the locked position indicator indica 230c. In this position, a retention shaft 240 (including a split distal end 242 thereof) is locked within the locking interface 216 of a cannula 214, and the split distal end 242 and removeable tip 247 are locked in place, as shown in FIGS. 50 and 51.

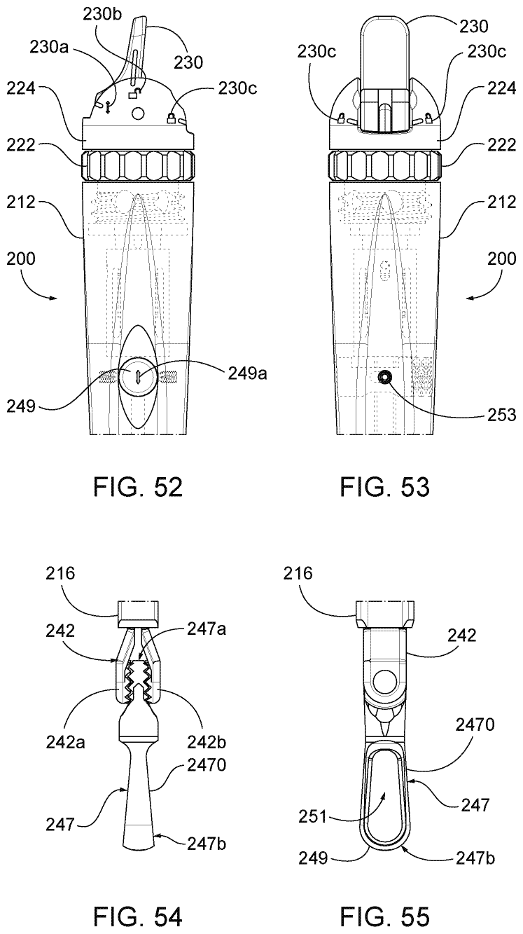

FIGS. 52 and 53 show the pivot lock mechanism 224 of the surgical instrument 200 with the lock lever 230 rotated to an unlocked position, as denoted by the unlocked position indicator indica 230b. In this position, the retention shaft 240 is moved distally away from the locking interface 216 of the cannula 214 and pivot lock mechanism 224, as shown in FIGS. 54 and 55. As with the surgical instrument 10 described above, this distal movement allows the split distal end 242 to open, and therefore allow the articulating tip to articulate. In other words, in this position, the portions 242a and 242b of the distal end 242 splay apart enough to allow articulation of the tip 247 but do not splay open enough for removal of the tip 247. Like the tips of the embodiments discussed above, the tip 247 includes proximal and distal ends 247a, 247b, respectively.

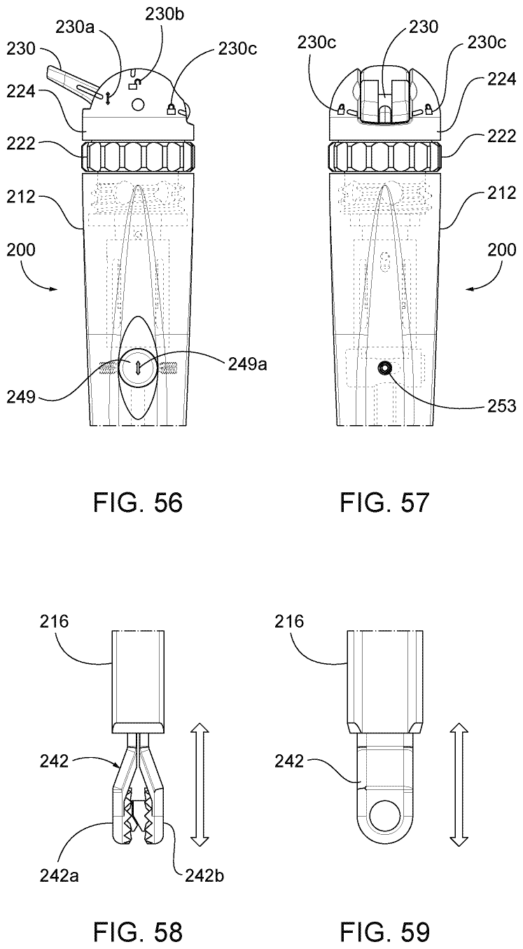

FIGS. 56 and 57 show the pivot lock mechanism 224 of the surgical instrument 200 with the lock lever 230 rotated to a retention shaft release position, as denoted by the retention shaft release position indicator indica 230a (i.e., a double ended arrow). The surgical instrument 200 also includes a retention shaft release button 249 with which must be depressed to enable the rotation of the lock lever 230 to the retention shaft release position. The button 249 is operably engaged with the sliding member (not shown, but the same as the sliding member 34 of the first embodiment) of the pivot lock mechanism 224, whereby depressing the button 249 allows the sliding member 234 to move distally as the lever 230 is moved to the retention shaft release position and release the retention shaft 240 (and the split distal end 242 thereof), as shown by the arrows in FIGS. 58 and 59. Like the retention shaft release position indicator indica 230a, the button 249 also includes retention shaft release indicia 249a thereon. The surgical instrument 200 also includes a dog-point set screw 253 that is used to capture and limit the travel of the release button 249. Other suitable means for capturing and limiting travel of the release button may be used in alternate embodiments.

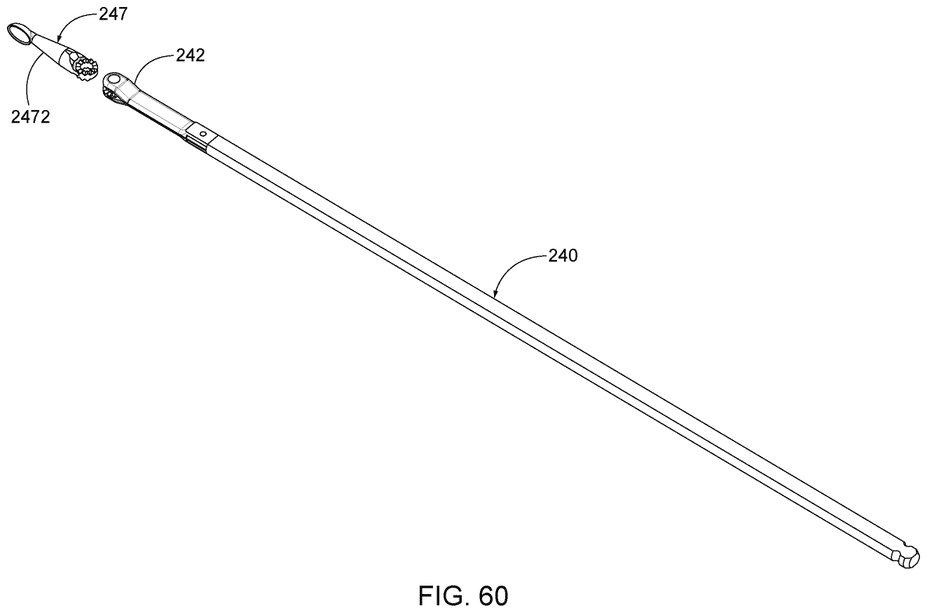

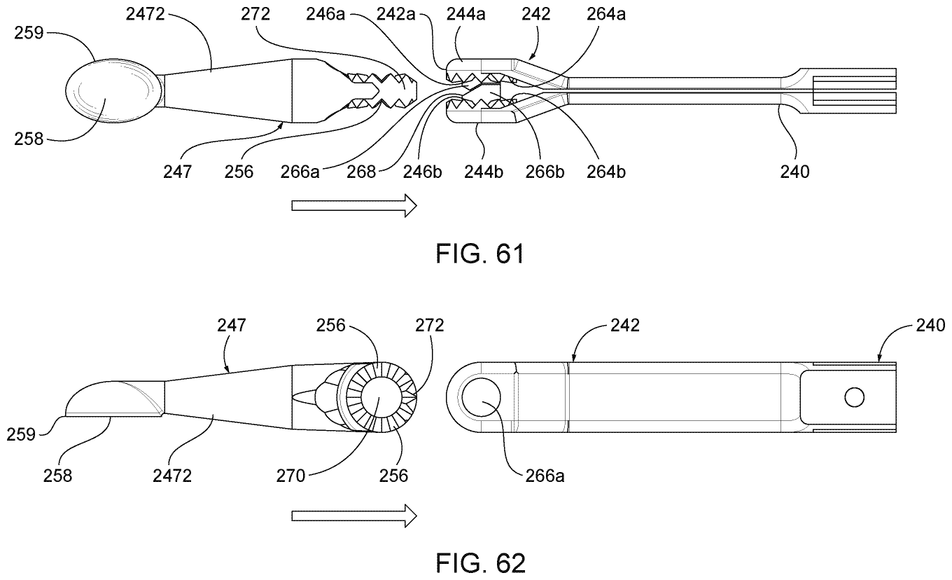

FIG. 60 shows the retention shaft 240, including its split distal end 242 and the removeable tip 247 of the third embodiment of the surgical instrument 200. FIGS. 61-64 are more detailed views of the split distal end 242 and tip 247, and are further discussed below.

The split distal end 242 of the retention shaft 240 includes first and second portions 242a, 242b, each having a respective outer surface 244a, 244b and inner surfaces 246a, 246b. The inner surfaces 246a, 246b each have teeth 264a, 264b and pins, or posts, 266a, 266b extending therefrom. The teeth 264a, 264b surround the respective pins 266a, 266b, as shown in FIGS. 61, 63 and 64. The pins 266a, 266b also define a chamfer 268, to facilitate interconnection of the split distal end 242 and removable tip 247, as further discussed below.

A removeable tip 247 according to this embodiment is shown in FIGS. 61-64. The tip 247 has a cup curette configuration 2472 and includes a debridement feature 258 at its distal end, and teeth 256 formed in a ring at its proximal end. The ring includes teeth 256 on both superior and inferior surfaces of the proximal end and defines a pivot aperture 270 surrounded by the teeth 256. The teeth 256 also define a chamfer 272 that engages with the chamfer 268 to facilitate interconnection of the split distal end 242 and tip 247. The pivot aperture 270 is configured to rotatably engage/receive the pins 266a, 266b therein.

In operation, the proximal end of the removeable tip 247 is inserted (i.e., pushed) into the split distal end 242, splaying apart the first and second portions 242a, 242b thereof. The chamfer 272 of the teeth 256 on the tip 247 and the chamfer 268 of the pins 266a, 266b of the split distal end 242 facilitate the splaying and insertion. Once the proximal end of the removeable tip 247 has been inserted into the split distal end 242, the first and second portions 242a, 242b snap back together (i.e., return to their non-splayed configuration), whereby the respective pins 266a, 266b of the first and second portions 242a, 242b insertably engage the pivot aperture 270 of the tip 247 and retain the tip 247 within the split distal end 242. The pins 266a, 266b and pivot aperture 270 are designed to allow a simple push-together assembly of the tip 247 and split distal end 242.

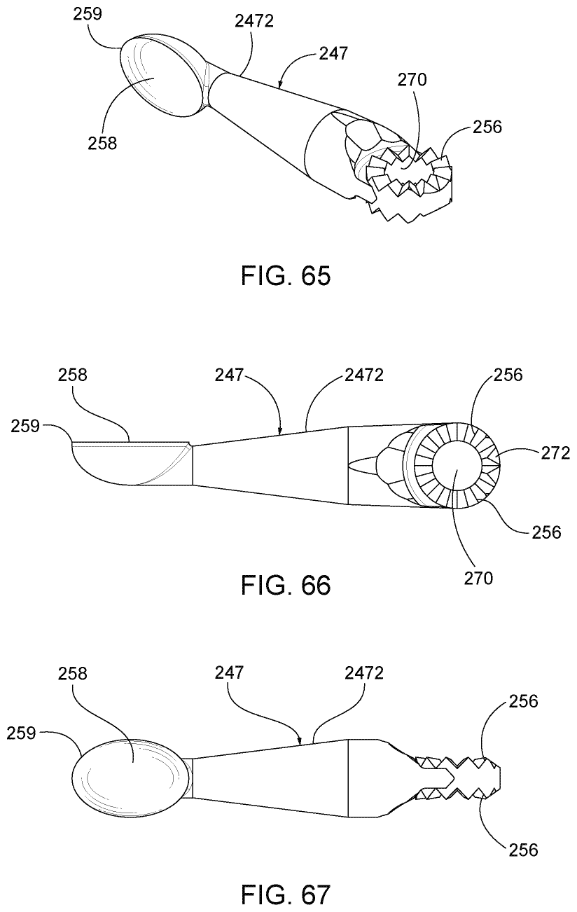

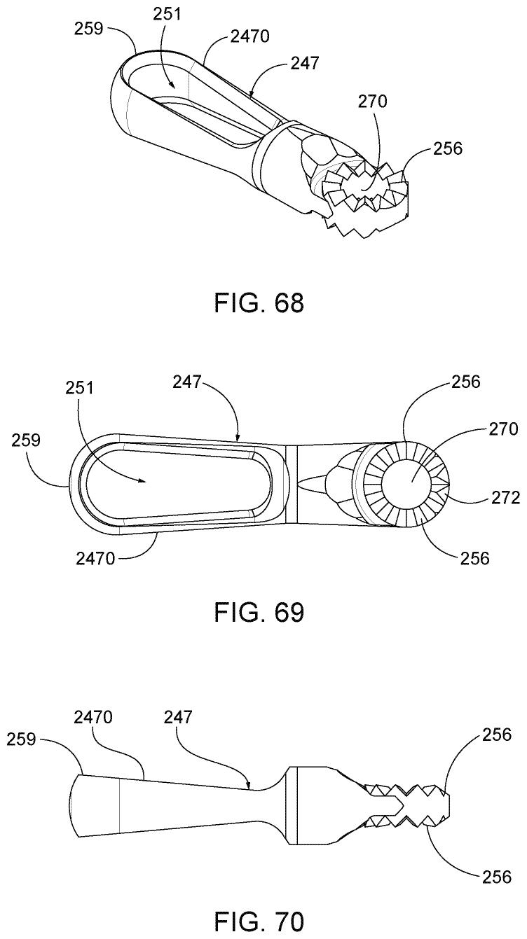

Embodiments of removeable tips 247 configured as cup curettes 2472 are shown in FIGS. 65-67 and include a debridement feature 258 and a cutting edge 259 (i.e., for scraping bone or other tissue during surgery) at its distal end (opposite the teeth 256 thereof). In some embodiments (not shown), the cup curette 2472 has a serrated configuration. Other embodiments of removeable tips 247 that are configured as stirrup curettes 2470 are shown in FIGS. 68-70 and include the opening 251 and cutting edge 259.

An alternate embodiment of the removeable tip 347 is shown in FIGS. 71 and 72. Here, the removeable tip includes a rasp configuration 360 that includes a plurality of pointed projections 363 for use in scraping and removing tissue during surgery.

Components of the embodiments of the invention are formed from materials having properties that enable the components to perform their respective functions. Such materials are biocompatible, and include, for example, stainless steels. The material from which the split distal end of the retention shaft is formed must possess a sufficient resiliency so as to imbue this element with the level of flexibility required for its operation, as described above. Such materials include, for example, stainless steels.

In various embodiments, the components of the invention may be provided in a surgical kit. The kit may include the surgical instrument as described herein, along with a plurality of interchangeable removeable tips for use in surgery, as also described herein.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other examples are also within the claims.

In general, any combination of disclosed features, components and methods described herein is possible. Steps of a method can be performed in any order that is physically possible.

All cited patents, patent publications and non-patent references are incorporated by reference herein in their entireties.

Although embodiments have been disclosed, the invention is not limited thereby.

What is claimed is:

1. A surgical instrument comprising:
a handle having a proximal end, a distal end, and a cannula;
a retention shaft disposed within and configured to engage a distal end of the cannula, the retention shaft including a split distal end having a first portion and a second portion;
a removeable tip configured to be inserted in the split distal end between the first portion and the second portion;
a lock lever disposed at the proximal end of the handle, a linkage connected to the lock lever, a sliding member connected to the linkage and the retention shaft, wherein the linkage and sliding member are configured to translate rotational motion of the lock lever to linear motion of the retention shaft so that the retention shaft moves axially within the cannula;
wherein the lock lever is moveable between a locked position in which the removeable tip is not pivotable, an unlocked position in which the removable tip is pivotable, and a retention shaft release position in which the retention shaft is released from the sliding member, the retention shaft being engaged with the distal end of the cannula to prevent the removable tip from pivoting when the lock lever is in the locked position; and
wherein the surgical instrument further includes a retention shaft release button, whereby the retention shaft cannot be released from the linkage until the button is engaged.

2. The surgical instrument of claim 1, wherein the removeable tip is secured between the first and second portions by a friction fit.

3. The surgical instrument of claim 1, wherein the removeable tip is secured between the first and second portions by teeth formed on the inner surfaces of the first and second portions.

4. The surgical instrument of claim 1, wherein the removeable tip has a configuration selected from a cup curette configuration and a stirrup curette configuration.

5. The surgical instrument of claim 1, wherein the removeable tip has a rasp configuration.

6. The surgical instrument of claim 1, wherein the lock lever is a pivoting lock lever.

7. The surgical instrument of claim 1, wherein the removeable tip includes a distal end and a proximal end, the proximal end of the removable tip being configured to removably engage the split distal end of the retention shaft.

8. The surgical instrument of claim 7, wherein the proximal end of the removeable tip includes at least one pin configured to engage at least one opening of the split distal end, and teeth arranged to engage and interlock with teeth on the split distal end.

9. The surgical instrument of claim 7, wherein the proximal end of the removeable tip includes teeth formed in a ring, and the split distal end includes teeth.

10. The surgical instrument of claim 7, wherein the split distal end has a first chamfer and the removeable tip has a second chamfer that engages with the first chamfer to facilitate insertion of the removable tip in the split distal end.

11. The surgical instrument of claim 10, wherein the split distal end splays open to allow insertion of the removable tip through the interaction of the first chamfer and the second chamfer.

12. The surgical instrument of claim 1, wherein the removable tip is one of a plurality of interchangeable removeable tips configured to be inserted in the split distal end between the first and second portions.

13. The surgical instrument of claim 10, wherein the interchangeable removeable tips each have a configuration selected from the group consisting of a cup curette configuration, a stirrup curette configuration and a rasp configuration.

14. The surgical instrument of claim 1, further comprising retention balls engaged with the retention shaft to limit the movement of the retention shaft.

15. The surgical instrument of claim 14, wherein pressing the button releases the retention balls from the retention shaft.

16. The surgical instrument of claim 15, wherein the lock lever is movable to a position for removing the removable tip from the split distal end, without releasing the retention shaft.

17. The surgical instrument of claim 1, wherein in the unlocked position of the lock lever the split distal end of the retention shaft splays open to allow the removable tip to pivot, and wherein the lock lever has a third position, and wherein the split distal end splays open to allow removal of the removable tip in the third position.

18. The surgical instrument of claim 17, wherein the retention shaft release position is a fourth position of the lock lever.
